(19) United States
(12) Patent Application Publication
(10) Pub. No.: US 2025/0269135 A1
(43) Pub. Date: Aug. 28, 2025

(54) BREATHING GAS HUMIDIFIER WITH A LIQUID CONTAINER AND A LOCKING UNIT

(71) Applicant: Drägerwerk AG & Co. KGaA, Lübeck (DE)

(72) Inventor: Markus HAMPE, Lübeck (DE)

(21) Appl. No.: 19/059,527

(22) Filed: Feb. 21, 2025

(30) Foreign Application Priority Data
Feb. 22, 2024 (DE) 10 2024 104 971.5

(51) Int. Cl. A61M 16/10

(52) U.S. Cl. CPC A61M 16/109

(57) ABSTRACT

A breathing gas humidifier includes an exchangeable container (10) for a liquid to be evaporated, in particular for water. The container can be inserted into a receptacle of a housing and removed again from the receptacle. A heating element heats the liquid in the container (10). In a locking position, a locking unit (3, 26) locks the container (10) in the receptacle. With the locking unit (3, 26) in a releasing position, the container (10) can be removed from the receptacle. A connecting unit (35, 37) mechanically connects the locking unit to the housing. During the movement from one position to the other position, the connecting unit (35, 37) guides the locking unit (3, 26) along a trajectory extending in a plane. Preferably, the connecting unit (35, 37) includes two spaced-apart connecting elements (35, 37) and that connect the locking unit to the housing as a four-bar linkage.

### CROSS REFERENCE TO RELATED APPLICATIONS

This application claims the benefit of priority under 35 U.S.C. § 119 of German Application 10 2024 104 971.5, filed Feb. 22, 2024, the entire contents of which are incorporated herein by reference.

### TECHNICAL FIELD

The present invention relates to a breathing gas humidifier (respiratory humidifier) with an exchangeable (replaceable) container for a liquid to be evaporated and/or vaporized, in particular for water, and with a locking unit for locking the container.

### BACKGROUND

In the following, a “breathing gas humidifier” is understood to mean a device which is configured to evaporate a liquid and thereby increase the content of the evaporated liquid in a gas mixture. In particular, the liquid is water or another liquid that can be added to a gas mixture that is inhaled by a person. The gas mixture contains oxygen and can be inhaled by a person.

In particular, such a breathing gas humidifier is used during artificial ventilation (ventilation) of a patient. A patient-side coupling unit is arranged on and/or in the patient, which comprises, for example, a breathing mask or a tube. A ventilator or other medical device expels the breathable gas mixture. The expelled gas mixture is guided through a fluid guide unit to the patient-side coupling unit. Preferably, the fluid guide unit comprises a hose. The gas mixture contains oxygen and, in one embodiment, additionally at least one anesthetic agent, so that the patient is sedated or even anaesthetized, and/or a drug (medication).

The patient inhales the gas mixture, for example due to his/her own respiratory activity and/or due to ventilation strokes from the ventilator. Therefore, the inhaled gas mixture inevitably comes into contact with parts of the patient's body, such as the nasal mucosa and/or lung tissue. It must be prevented that these parts of the body are dried out by the inhaled gas mixture. Therefore, the content of water vapor in the gas mixture is sufficiently increased before the gas mixture reaches the patient-side coupling unit.

As a rule, such a breathing gas humidifier works as follows: The breathing gas humidifier includes a container, often called an evaporator unit or humidifier chamber. This container contains a liquid that comprises liquid water. In the following, the term water is used as a representative term for the liquid to be evaporated.

A heating element of the breathing gas humidifier heats the water in the container so that at least some of the water is vaporized and thus evaporates. The gas mixture, whose water vapor content is to be increased, is fed into the container and absorbs water vapor there. The gas mixture with the increased water vapor content is fed to the patient-side coupling unit. The breathing gas humidifier according to the invention also works according to this principle when used with water as the liquid.

As a rule, the container is usually locked in a specific position in relation to a housing of the breathing gas humidifier. It is usually necessary to remove the container from the breathing gas humidifier from time to time and reinsert the same or a different container. For example, a container is only used for a certain time period or until the liquid to be evaporated has been used up and the container is then disposed of. Or liquid must be filled into the container, or the container must be cleaned and/or disinfected.

### SUMMARY

It is an object of the invention to provide a breathing gas humidifier with a receptacle for a container with a liquid to be evaporated, in which receptacle the container can be replaced relatively easily.

The problem is solved by a breathing gas humidifier with features according to the invention. Advantageous embodiments are presented in this disclosure.

The term “breathing gas humidifier” has already been defined above. The breathing gas humidifier according to the invention comprises a container which holds a liquid to be evaporated and/or vaporized. Preferably, a gas mixture flows through the container and is to be enriched with evaporated and/or vaporized liquid as it flows through, this liquid originating from the container. The container comprises a chamber for holding the liquid and, in one embodiment, a circumferential protruding rim around its underside. In one embodiment, the container has the shape of a cylinder.

Furthermore, the breathing gas humidifier according to the invention comprises a housing. The housing comprises a receptacle for the container and a heating element. The heating element is preferably located outside the container and is preferably recessed in the housing. The receptacle is, for example, a recess in the housing.

The container with the liquid can be inserted into the receptacle. The container can be removed from the receptacle. If the container is correctly inserted into the receptacle, the container is in thermal contact with the heating element. The heating element is able to supply thermal energy to the liquid in the container and, as a result, evaporates and/or vaporizes liquid in the container. This results in evaporated and/or vaporized liquid, for example water vapor, in the container.

A locking unit of the breathing gas humidifier according to the invention can be moved back and forth relative to the receptacle between a locking position and a releasing position. Preferably, the locking unit is a purely mechanical component, i.e. in particular it does not require a supply of electrical or pneumatic or hydraulic energy or an electronical actuation or control. In the locking position, the locking unit holds the container with the liquid in the receptacle. In this way, the container is locked in the receptacle and movement of the container away from the receptacle is prevented. Preferably, any linear movement of the container relative to the receptacle is prevented, and no movement at all or only a rotational movement relative to the receptacle is still possible. In the releasing position, the locking unit enables the container to be removed from the receptacle and the same container or another container to be inserted into the receptacle. After the container has been inserted into the receptacle, the locking unit is moved back into the locking position. Thereby, the locking unit locks the inserted container.

According to the invention, a connecting unit mechanically connects the locking unit to the housing. The connecting unit is also preferably a purely mechanical component. The mechanical connection provided by the connecting unit makes it possible to move the locking unit from one position to the other position. The mechanical connection is configured in such a way that the following desired effect is achieved: While the locking unit is moved from one position to the other position, the locking unit is guided relative to the housing along a trajectory. This trajectory preferably extends in a plane. In one embodiment, this plane is arranged vertically, i.e. points to the center of the earth, provided that the humidifier stands on a level (even) surface. Preferably, the releasing trajectory along which the locking unit is moved from the locking position to the releasing position points diagonally downwards. The locking trajectory, along which the locking unit is moved from the releasing to the locking position, is opposite to the unlocking trajectory.

According to the invention, the container with the liquid to be evaporated and/or vaporized can be inserted into the receptacle. The inserted container is locked in the locking position by the locking unit. In many cases, the interaction (cooperation) between the locking unit and the housing prevents the container from moving relative to the receptacle. Such a movement is undesirable in many cases. This is because a leak could occur as a result of such a movement. A gas mixture could escape from such a leak. This gas mixture may contain the evaporated liquid. In the case of artificial ventilation, the gas mixture may also contain at least one anesthetic and/or a drug. It is therefore generally undesirable for a gas mixture to escape from a leak into the environment. In addition, in many cases a leak can lead to less liquid being evaporated or vaporized than required for the same heating capacity per unit of time.

If the container moves relative to the receptacle, the following undesirable event can also occur: The container loses thermal contact with the heating element at least temporarily, or at least the heat conduction and thus the amount of heat and thermal energy transferred is reduced. As a result, the heating element is able to evaporate and/or vaporize less liquid in the container, in extreme cases even no liquid at all. This effect is also undesirable. In extreme cases, the event that the container moves relative to the receptacle can even lead to the container falling out of the receptacle and being damaged.

According to the invention, the connecting unit mechanically connects the locking unit to the housing. While the locking unit is moved from one position to the other position, the locking unit is guided along a trajectory. The connecting unit ensures that the locking unit is only moved along this trajectory, i.e. is guided, and does not leave this trajectory. The trajectory lies in one plane. Because the locking unit is guided during the movement, the risk of the moving locking unit touching a surface of the receptacle or a surface of another part of the housing of the breathing gas humidifier or a surface of the container and scratching along this surface is reduced. This also reduces the risk of the locking unit jamming or canting during movement. In many cases, it is ensured that there is always a sufficiently large gap between a surface of the housing and the moving locking unit.

According to the invention, the locking unit can be moved back and forth between a locking position and a releasing position. In the locking position, the locking unit holds the container in the receptacle. Preferably, the locking unit comprises a locking component, i.e. a component that comes or can come into contact with the inserted container, in particular a locking body. Preferably, a resetting element, in particular at least one mechanical or pneumatic spring, strives to move the locking unit into the locking position and to hold it in the locking position. Alternatively or additionally, a latching connection or snap connection ensures that the locking unit remains in the locking position. In particular, these two possible implementations result in a secure state being established without external intervention and reduce the risk of the locking unit being moved unintentionally into the releasing position. The configuration with the resetting element also reduces the risk that a container inserted into the receptacle is unintentionally not locked.

Preferably, in addition to the locking component, the locking unit comprises an actuating component that is mechanically connected to the locking component. Preferably, a user can move the locking unit into the releasing position by means of the actuating component. For example, the user actuates the actuating component and thereby moves the locking unit into the releasing position against the restoring force of the resetting element. In one embodiment, the actuating component and the locking component form a single component. It is also possible for the user to release a latching connection or snap connection using an actuating element and then move the locking unit into the releasing position. This actuating element can be spatially spaced from the locking body.

According to the invention, the connecting unit mechanically connects the locking unit to the housing of the breathing gas humidifier. The connecting unit ensures that the locking unit can only be moved relative to the housing along a trajectory extending in a plane from one position to the other position, thus guiding the locking unit. In a preferred embodiment, the connecting unit comprises a first connecting element and a second connecting element. The two connecting elements are spatially spaced apart. Each connecting element is connected to both the housing and the locking unit in a pivotable (articulated), in particular rotatable, manner.

The first connecting element is connected to the housing at at least two connection points and to the locking unit at at least two further connection points. The second connecting element is connected to the housing in at least one connection point, preferably in at least two connection points, and to the locking unit in at least one further connection point, preferably in at least two further connection points. A total of at least six, preferably at least eight connection points are thus provided, namely three, preferably four connection points for the mechanical connection between the connecting unit and the housing and three, preferably four further connection points for the mechanical connection between the connecting unit and the locking unit. Thanks to these six, preferably eight, connecting points, the connecting unit is able to guide the locking unit along the trajectory in a particularly secure manner.

Preferably, each connecting element is rotatably connected to the housing and rotatably connected to the locking unit. This implementation provides a total of eight connection points. Two connection points each define an axis of rotation. A total of four axes of rotation are therefore formed. According to this implementation, each connecting element is rotatably connected to the housing about one of the four axes of rotation. The two axes of rotation, in which a connecting element is connected with the housing and with the locking unit, resp., are arranged parallel to each other. Preferably, there is a distance between these two parallel axes of rotation. Particularly preferably, all four axes of rotation are parallel to each other and spaced apart in pairs.

Particularly preferably, the two connecting elements connect the locking unit to the housing in such a way that the following is achieved: The locking unit is connected to the housing by a four-bar linkage (articulated quadrangle, hinged quadrilateral). The geometry of this four-bar linkage changes while the locking unit is moved relative to the housing. The side lengths of the four-bar linkage remain constant, the internal (inside) angles of the four-bar linkage change.

The configuration with the two spaced-apart connecting elements and in particular the configuration with the four-bar linkage further reduce the risk of the locking unit tilting or canting or jamming during a movement from one position to the other or coming into contact with a surface of the housing or with a surface of the container and scratching along this surface. Rather, the reliability is further increased that the locking unit is actually (indeed) guided along a desired trajectory, preferably a planar (flat, even) trajectory. The embodiment just described therefore increases the operational reliability of the humidifier compared to other possible embodiments of the connecting unit.

The following describes various ways in which these two connecting elements are configured and, in particular, how they implement a four-bar linkage.

In one embodiment, the first connecting element comprises a U-shaped bracket with two legs and a connecting rod on each of the two legs, i.e. a total of two connecting rods. Preferably, the two legs are arranged at the two ends of a middle part (central section), wherein the middle part is preferably rod-shaped and wherein the middle part is located between the two connecting rods. Particularly preferably, the two connecting rods point outwards, i.e. away from the middle part. The first connecting element is inherently torsionally rigid (torsion-resistant). In particular, the bracket cannot be twisted. The two connecting rods are rotatably connected to the housing in such a way that the first connecting element can rotate relative to the housing, but preferably cannot move parallel or at an angle to this axis of rotation.

This configuration further reduces the risk of the locking unit tilting or canting while it is moved from one position to the other. It is often sufficient to press against the locking unit at one point in order to move the locking unit without tilting. It is possible, but thanks to the torsional rigidity not necessary, to press against the locking unit at two points which are spaced apart from each other. It is even possible that this one point is relatively far away from the plane in which the trajectory of the movement of the locking unit extends.

Preferably, the second connecting element is also rotatably connected to the housing. Particularly preferably, the second connecting element cannot move relative to the housing parallel or at an angle to the axis of rotation.

In one embodiment, the second connecting element comprises a guide element on the connecting element side. The housing comprises a guide element on the housing side. The guide element on the connecting element side corresponds (cooperates) with the guide element on the housing side. Thanks to this correspondence, the second connecting element is guided along a connecting element trajectory while the locking unit is moved from one position to the other. For example, one guide element engages with the other guide element and the two guide elements move relative to each other while the locking unit is moved from one position to the other position.

In a further embodiment, the locking unit comprises a guide element on the locking unit side. The guide element on the connecting element side just mentioned corresponds (cooperates) with the guide element on the locking unit side. In particular, one guide element engages with the other guide element. Thanks to this correspondence, the second connecting element is guided along a connecting element trajectory while the locking unit moves from one position to the other.

The two implementations just described can be combined with each other. According to this combination, the housing comprises a guide element on the housing side, the locking unit comprises a guide element on the locking unit side and the second connecting element comprises a guide element on the connecting element side. The guide element on the connecting element side corresponds (cooperates) both with the guide element on the housing side and with the guide element on the locking unit side. The combination of these implementations causes the locking unit to be guided along the connecting element trajectory while moving from one position to the other.

Each of the implementations just described, in which the locking unit is additionally guided, increases the reliability that the locking unit is actually guided along the desired trajectory extending in a plane and is not laterally offset or displaced or twisted or tilted or cants.

According to the invention, the locking unit is movably connected to the housing. The connecting unit, which movably connects the locking unit to the housing, defines which orientation relative to the housing the locking unit has in the locking position and which orientation relative to the housing it has in the releasing position. In a preferred embodiment, the locking unit comprises a plate. Preferably, this plate acts as an actuating component (actuator), and a human can move or otherwise actuate this actuating component to move the locking unit. Preferably, a locking body of the locking unit is connected to this plate, in particular fixedly connected.

In one embodiment, the connecting unit connects the locking unit to the housing in such a way that the following is achieved: If the locking unit is in the locking position, the plate rises (ascends), namely with respect to a horizontal viewing direction towards the receptacle.

The rising orientation of the plate reduces the risk that the container with the liquid to be evaporated and/or vaporized is brought into the vicinity of the receptacle or even inserted into the receptacle, but is not locked in place. As a result of an unlocked state, the container can change its position relative to the receptacle during use, which event is generally undesirable as explained above. The embodiment with rising orientation of the plate reduces the risk of this undesirable event as follows: In many cases, if the container is not properly locked in place, the container will slide down over the plate at an angle (with rising orientation) and slide away from the receptacle. A user usually notices this event.

In a further embodiment, the connecting unit connects the locking unit to the housing in such a way that, in one embodiment, the following is achieved: If the locking unit is in the releasing position, the plate is arranged horizontally. In another embodiment, the following is achieved: The plate, and particularly preferably the entire locking unit, is located at an angle below the container when the container is inserted into the receptacle and the locking unit is in the releasing position.

The horizontal arrangement of the plate makes it easier to remove the container from the receptacle, clean the receptacle if necessary and/or refill with liquid and insert the same or another container into the receptacle. In many cases the feature that the locking unit in the releasing position is located vertically or at an angle below the receptacle makes it easier to quickly remove the container from the receptacle.

If the locking unit is in the releasing position, it is particularly preferable that the plate of the locking unit is completely below the container when the container is inserted into the receptacle. In this implementation, the container can be removed from the receptacle by a linear movement over the plate. The same or another container can be inserted into the receptacle by a horizontal movement in the opposite direction. With this implementation, it is not necessary to lift the container to remove it from the receptacle.

These two configurations can be combined with each other. If the locking unit is in the locking position, the plate rises towards the receptacle (at an angle with respect to the receptacle) and if the locking unit is in the releasing position, the plate is horizontal.

The configuration in which the plate rises at an angle in the locking position and is horizontal in the releasing position can be implemented with particularly high reliability if the connecting unit comprises two connecting elements as described above. Reliability is further increased if the connecting unit provides a four-bar linkage as described above.

An embodiment was mentioned above in which a resetting element strives to move (is biased towards) the locking unit into the locking position and to hold the locking unit in this position. The embodiment with the resetting element can be combined with the embodiment just described, in which the locking unit comprises a plate. As a rule, the resetting element strives to move the locking unit angularly upwards against the force of gravity. According to the combination just described, it is possible in many cases to insert the container with a liquid into the receptacle as follows: The plate of the locking unit is pressed down with the aid of the container to be inserted, preferably with the container on the plate and pressed downwards. The container is inserted into the receptacle. At the latest when the container is correctly inserted into the receptacle and the locking unit is no longer pressed down, the following step is carried out automatically: The resetting element moves the locking unit, preferably upwards, and the container is locked in place. In many cases, this configuration eliminates the need to move the locking unit itself. Rather, it is sufficient to move the container, preferably until it no longer presses down the locking unit.

In one embodiment, the breathing gas humidifier comprises an ejector, which is preferably implemented as a mechanical or pneumatic spring unit. The ejector may additionally or instead comprise a controllable electrical or electromagnetic or pneumatic or hydraulic actuator. The ejector is supported by the receptacle. The ejector strives to move a container that is inserted into the receptacle away from the receptacle. In the releasing position, the locking unit is located completely vertically or diagonally below the container in the receptacle. The ejector then moves the container away from the receptacle. In the locking position, the locking unit prevents the ejector from moving the container away from the receptacle.

In many cases, this configuration makes it possible to remove the container from the receptacle with a single movement. It is sufficient to move the locking unit from the locking position to the releasing position. The ejector then moves the container out of the receptacle over the locking unit.

According to the invention, the container is received by the receptacle, and the locking unit in the locking position locks the container in the receptacle. In a preferred embodiment, the breathing gas humidifier is configured as follows: If the locking unit is in the locking position, the locking unit and the receptacle completely surround the container, except for unavoidable gaps and/or slits. This further reduces the risk of the container making an unwanted movement relative to the housing.

Preferably, a breathable gas mixture is supplied to the breathing gas humidifier by means of a feed (supply) fluid guide unit. The breathing gas humidifier should enrich this gas mixture with an evaporated or vaporized liquid, whereby this liquid preferably comprises water or is pure water. The enriched gas mixture is discharged via a discharge fluid guide unit. A “fluid guide unit” is understood to be a component which is capable of guiding a fluid along a trajectory, whereby the trajectory is predetermined by the configuration and/or by the arrangement of the component and whereby the component ideally prevents part of the fluid from leaving the trajectory. A hose and a tube are two examples of a fluid guide unit.

The invention also relates to a ventilation arrangement which is configured to artificially ventilate a patient. A patient-side coupling unit, for example a tube and/or a breathing mask, is arranged in and/or on the patient's body during artificial ventilation. A fluid guide unit establishes a fluid connection between the ventilator and the patient-side coupling unit. The ventilator is configured to expel a breathable gas mixture. The expelled gas mixture comprises oxygen and, in one embodiment, at least one anesthetic agent and/or at least one drug. The expelled gas mixture is guided through the fluid guide unit to the patient-side coupling unit and can be inhaled by the patient. A breathing gas humidifier according to the invention is capable of enriching the gas mixture expelled by the ventilator with an evaporated liquid, in particular with water vapor, alternatively with at least one anesthetic agent. The breathable gas mixture is enriched with this evaporated liquid before it reaches the patient-side coupling unit. Preferably, the breathing gas humidifier is arranged in the fluid guide unit.

Optionally, the patient-side coupling unit is also connected to the ventilator by a further fluid guide unit so that a ventilation circuit is provided. The air exhaled by the patient is fed to the ventilator through the additional fluid guide unit. If an anesthetic is fed to the patient during artificial ventilation, the configuration with the ventilation circuit reduces the risk of anesthetic escaping into the environment, which is generally undesirable. It also allows the ventilator to examine the air exhaled by the patient.

The receptacle for the container of the breathing gas humidifier can be integrated into a housing of the ventilator. Alternatively, the breathing gas humidifier forms a device that is spatially separated from the ventilator. Preferably, one segment of the fluid guide unit establishes a fluid connection between the ventilator and the breathing gas humidifier. Another segment of the fluid guide unit establishes a fluid connection between the breathing gas humidifier and the patient-side coupling unit.

The invention is described below by means of embodiment examples. The various features of novelty which characterize the invention are pointed out with particularity in the claims annexed to and forming a part of this disclosure. For a better understanding of the invention, its operating advantages and specific objects attained by its uses, reference is made to the accompanying drawings and descriptive matter in which preferred embodiments of the invention are illustrated.

### BRIEF DESCRIPTION OF THE DRAWINGS

In the drawings:

FIG. 1 is a schematic view of a ventilation arrangement with the humidifier of the embodiment example;

FIG. 2 is a perspective view of a humidifier with an evaporator unit;

FIG. 3 is a front view of a section of the humidifier shown in FIG. 2;

FIG. 4 is a perspective view from below showing the evaporator unit and the locking unit in the locking position;

FIG. 5 is a perspective view of the humidifier from below, with the evaporator unit and the locking unit omitted;

FIG. 6 is a sectional view from the side showing the evaporator unit and the locking unit in the locking position;

FIG. 7 is a sectional view from the side showing a detail of the illustration in FIG. 6;

FIG. 8 is a sectional view from the side showing the evaporator unit and the locking unit with the locking unit in the releasing position;

FIG. 9 is a sectional view from the side showing the ejected evaporator unit;

FIG. 10 is a sectional view from the side showing a detail of the illustration in FIG. 8; and

FIG. 11 is a detailed illustration of how the locking unit is guided.

### DESCRIPTION OF PREFERRED EMBODIMENTS

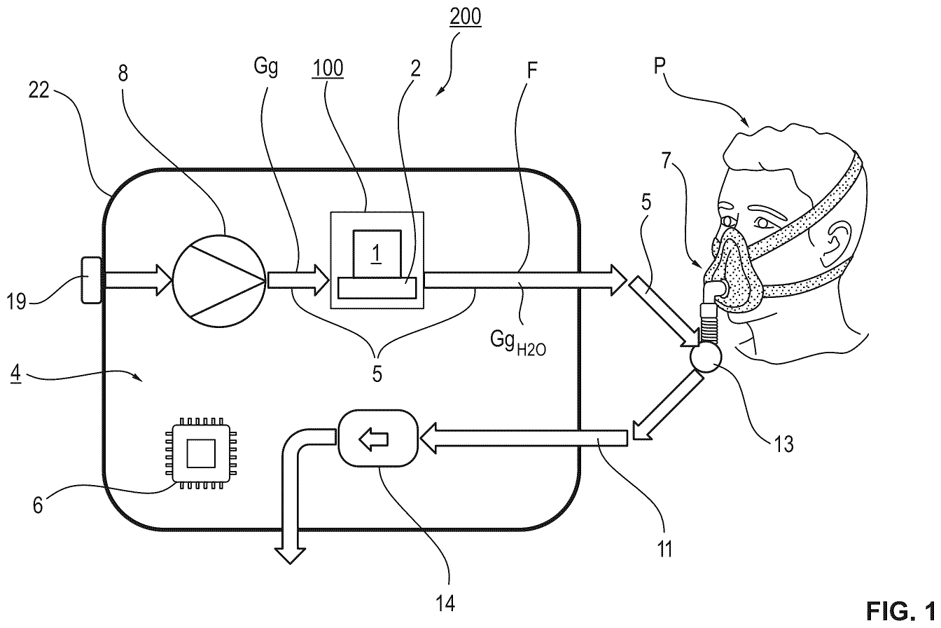

Referring to the drawings, FIG. 1 schematically shows a ventilation arrangement 200 which is capable of artificially ventilating a patient P. A medical device in the form of a ventilator 4 performs a sequence of ventilation strokes. The ventilator 4 is supplied with oxygen, breathing air and optionally anesthetic through a feed connection 19. A fluid conveying unit 8, for example a pump or a blower or a fan or a piston-cylinder unit, causes the ventilator 4 to expel in each ventilation stroke a respective quantity of a breathable gas mixture Gg. The gas mixture Gg comprises oxygen and, in one application, additionally at least one anesthetic agent and/or a drug. An inspiratory fluid guide unit 5 guides the expelled gas mixture Gg, Gg_{H2O} to a breathing mask 7, in a conveying direction indicated by arrows. The breathing mask 7 belongs to a patient-side coupling unit of the embodiment example. The patient-side coupling unit can also include a mouthpiece and/or a tube (not shown). In the embodiment example, the air exhaled by the patient P is guided through an expiratory fluid guide unit 11 into the environment. A Y-piece 13 connects the fluid guide units 5, 11 with the patient-side coupling unit 7. A valve 14 in the expiratory fluid guide unit 11 ensures an end-expiratory pressure.

A breathing gas humidifier 100 according to the invention is arranged between the ventilator 4 and the patient-side coupling unit 7. The position of the breathing gas humidifier 100 in FIG. 1 is only to be understood as an example. The breathing gas humidifier 100 can also be arranged outside the ventilator 4. The breathing gas humidifier 100 enriches the gas mixture Gg with water vapor. A gas mixture Gg_{H2O} enriched with water vapor is fed from the breathing gas humidifier 100 to the patient-side coupling unit 7.

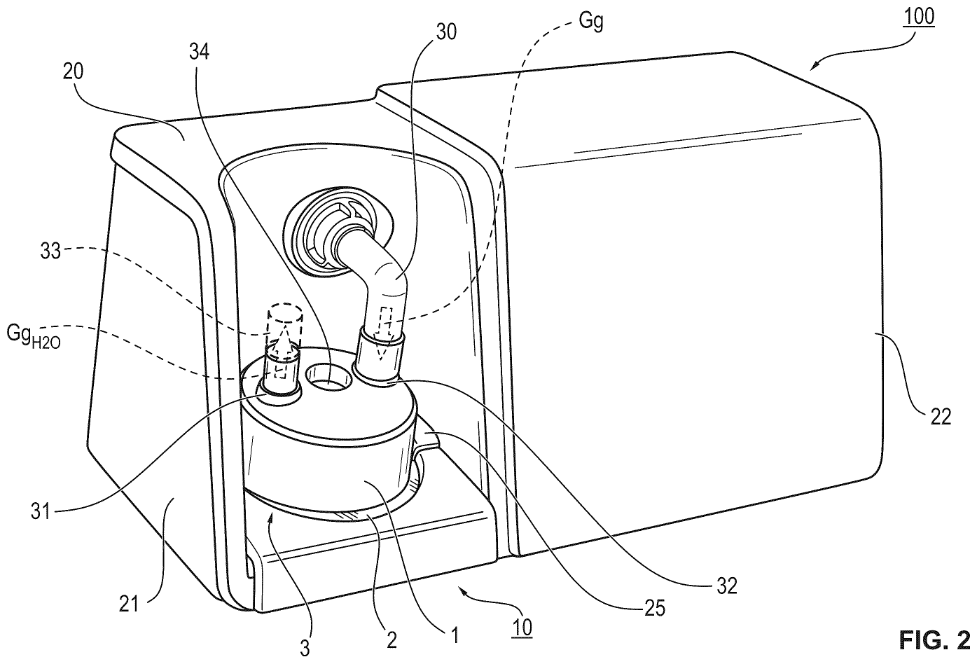

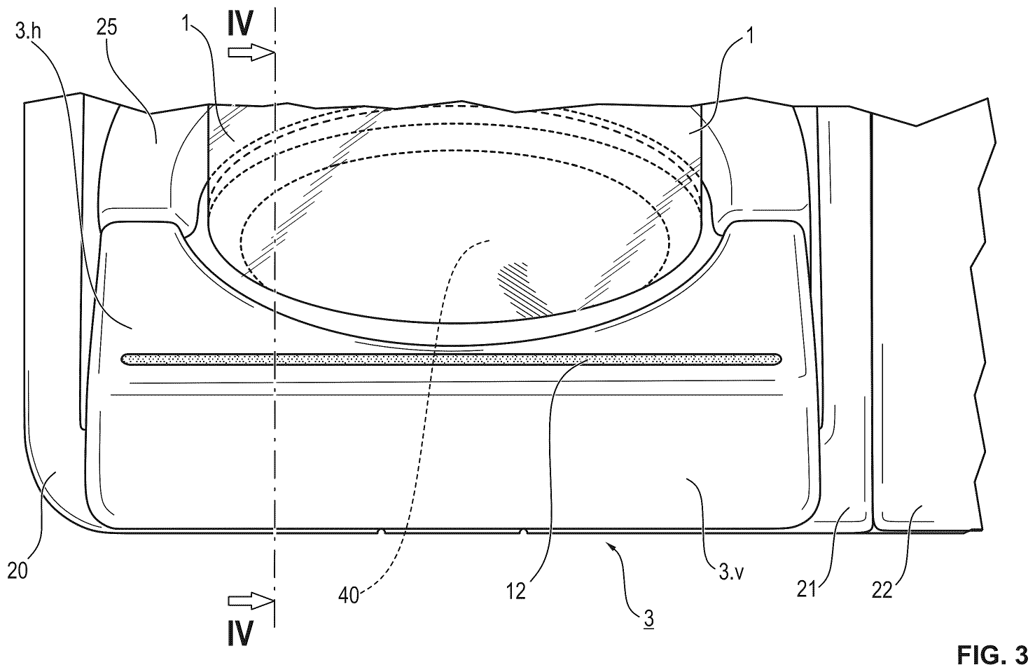

FIG. 2 shows an example of the breathing gas humidifier 100 in a perspective view obliquely from above and from the front. In this view, the breathing gas humidifier 100 is a component of the ventilator 4. FIG. 3 shows a detail of the breathing gas humidifier 100 of FIG. 2. The designations “above”, “below”, “front”, “rear”, “left” and “right” refer to the breathing gas humidifier 100 during use and to a viewing direction of a user looking at the breathing gas humidifier 100 from an operating side.

FIG. 2 schematically shows a housing 22 of the ventilator 4 and a two-part housing of the breathing gas humidifier 100. The housing 22 surrounds, among other things, a fluid conveying unit (not shown) in the form of a pump or a blower or a piston-cylinder unit. The housing of the breathing gas humidifier 100 comprises a main part 21 and a front part 20. The housing 20, 21 surrounds an infeed device and a control unit (both not shown). Preferably, the infeed device comprises an infusion tube which is guided into the evaporation chamber 1. The housing 20, 21 is fixedly connected to the housing 22. A semi-circular stop unit 25 is fixedly connected to the front part 21. In the embodiment example, the front part 20, the main part 21 and the stop unit 25 form a receptacle for an evaporator unit 10. This evaporator unit 10 comprises the container of the embodiment example.

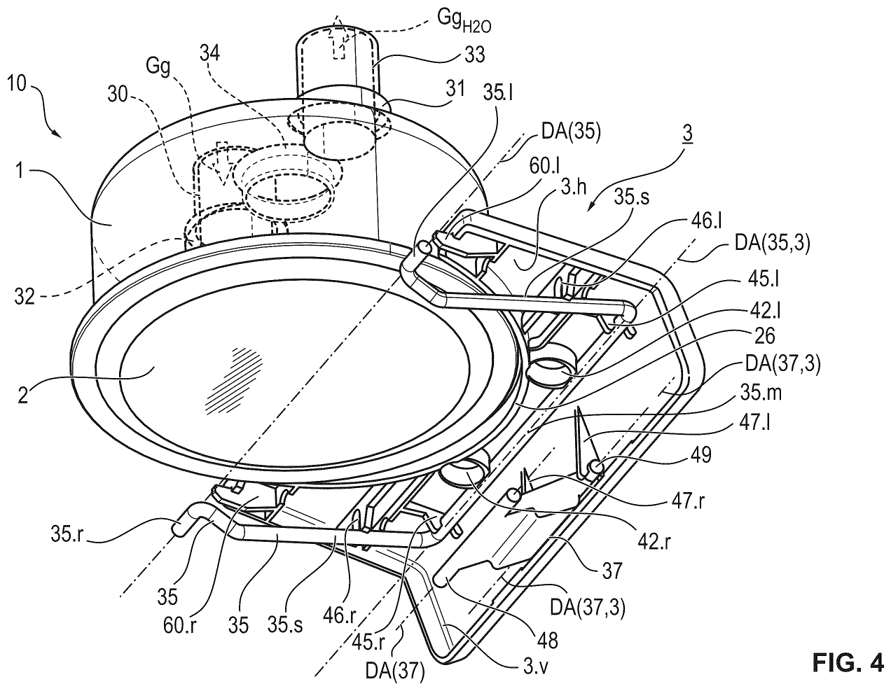

The evaporator unit 10 comprises a cylindrical evaporation chamber 1 and a circular base plate 2 and is rotationally symmetrical to a vertical axis of symmetry in the embodiment. The base plate 2 protrudes beyond the evaporation chamber 1 in all directions like a collar, which can be clearly seen in FIG. 4, for example. The evaporation chamber 1 can hold water. The infeed device is able to feed water into the evaporator unit 10.

A connection 32 for a feed fluid guide unit 30, a closable feed opening 34 for feeding in water and a connection 31 for a discharge fluid guide unit 33 are recessed into the cover of the evaporation chamber 1. The circular base plate 2 is adjacent to the semi-circular stop unit 25. The base plate 2 and thus the evaporator unit 10 are held in a specific position relative to the receptacle 20, 21, 25 by the stop unit 25 and by a semi-circular locking element 26. The locked evaporator unit 10 can perform a rotary movement about its own vertical axis of symmetry relative to the receptacle 20, 21, 25. The locking mechanism prevents any movement of the evaporator unit 10 away from the receptacle 20, 21, 25. In conjunction with the base plate 2, the locking mechanism also prevents the evaporator unit 10 from moving upwards, for example preventing movement of the evaporator unit 10 due to vibrations. The locking element 26 is part of a locking unit 3, 26, which is described in more detail below.

A retaining plate 40 with a heating element in the form of a heating plate and a retaining ring 41 is inserted into the main part 21. In one implementation, the heating plate is resiliently inserted into the retaining ring 41. When the evaporator unit 10 is inserted, the base plate 2 comes into thermal contact with the heating plate 40. The thermal contact is improved by the resilient mounting of the heating plate in the retaining ring 41, because the heating plate is pressed upwards against the base plate 2. When the heating plate 40 is heated and is in thermal contact with the base plate 2, the heating plate 40 causes water in the evaporator unit 10 to evaporate. A gas mixture Gg, which is fed into the interior of the evaporator unit 10 through the feed fluid guide unit 30, absorbs water vapor, thereby increasing the content of water vapor in the gas mixture Gg. The gas mixture Gg_{H2O} with the increased water vapor content is discharged (expelled) through the discharge fluid guide unit 33 and used in the embodiment example for artificial ventilation of the patient P.

The control unit of the breathing gas humidifier 100 is able to control the heating plate 40. The control task is to cause the heating plate 40 to generate a certain heat output. In one embodiment, the control unit carries out a closed-loop control. A sensor (not shown) measures the content (proportion, concentration, share) of water vapor in the gas mixture Gg_{H2O} downstream of the evaporator unit 10. Ideally, by controlling the heating plate, the control unit causes the actual water vapor content in the gas mixture Gg_{H2O} leaving the breathing gas humidifier 100 to be equal to a predetermined target water vapor content.

A level sensor (not shown) measures the fill level of water in the evaporator unit 10. If this fill level falls below a predefined threshold, the control unit generates a warning (alert).

The locking unit 3, 26 can be moved back and forth between a locking position and a releasing position. When the locking unit 3, 26 is in the locking position, the locking element 26 and the stop unit 25 almost completely surround the base plate 2. When the locking unit 3, 26 is in the releasing position, the evaporator unit 10 can be pulled away from the receptacle 20, 21, 25 and thus away from the heating plate 40, namely linearly forwards and thus towards the viewer of FIG. 2 and FIG. 3.

In one embodiment, a contact switch (not shown) detects the event that the locking unit 3, 26 is in the locking position. The same or another contact switch detects whether or not an evaporator unit 10 is correctly inserted into the receptacle 20, 21, 25. The level sensor mentioned above measures the fill level of water in the evaporator unit 10.

Preferably, the breathing gas humidifier 100 comprises at least one indicator element, preferably several indicator elements. These indicator elements indicate whether the above-mentioned three prerequisites for trouble-free operation of the breathing gas humidifier 100 are fulfilled or not, i.e. whether the evaporator unit 10 is correctly inserted, whether the locking unit 3, 26 is in the locking position, and whether there is sufficient water in the evaporator unit 10. In one embodiment, a display surface (display), which is not shown, is arranged on that surface of the housing 22 which faces the viewer in the representation of FIG. 2. The or each indicator element is implemented in a respective area of this display surface. Several indicator lights on the surface of the housing 22 are also possible. In an alternative implementation, an indicator light 12, shown only schematically, is mounted on the locking unit 3, 26, see FIG. 3. The representation of this indicator light 12 is to be understood only as an example.

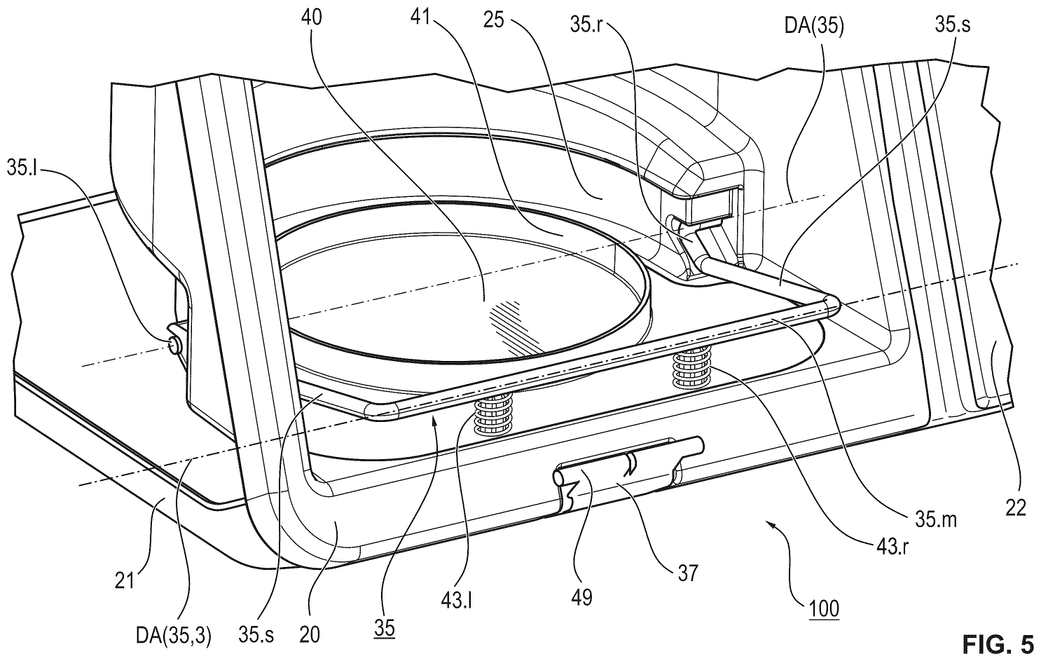

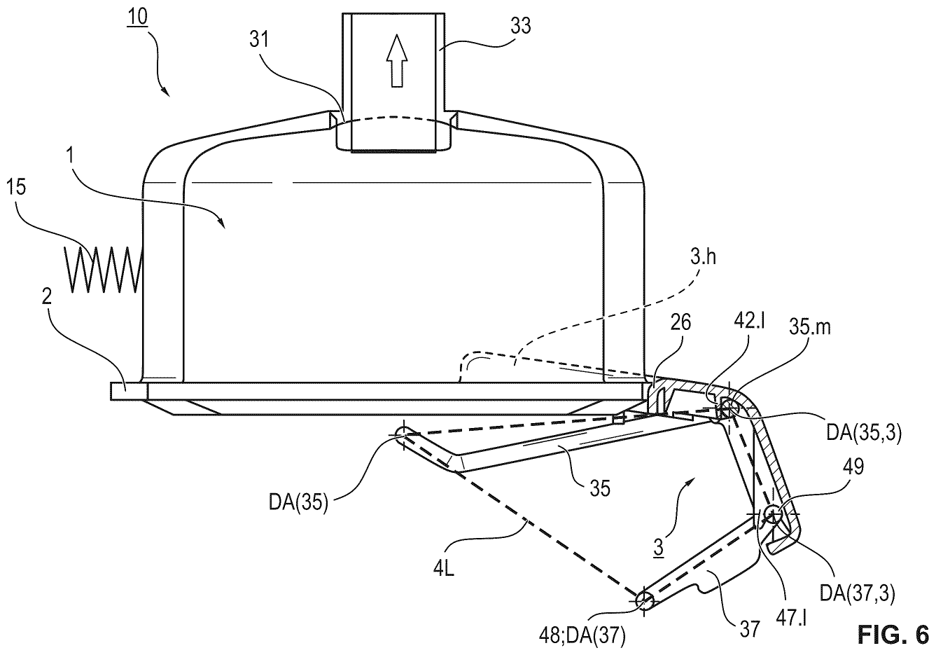

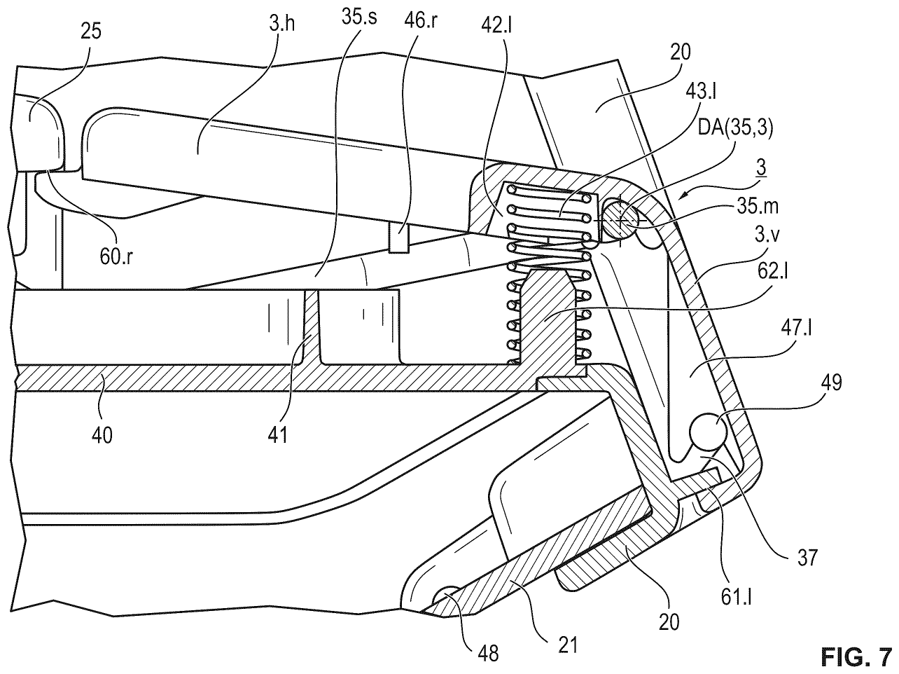

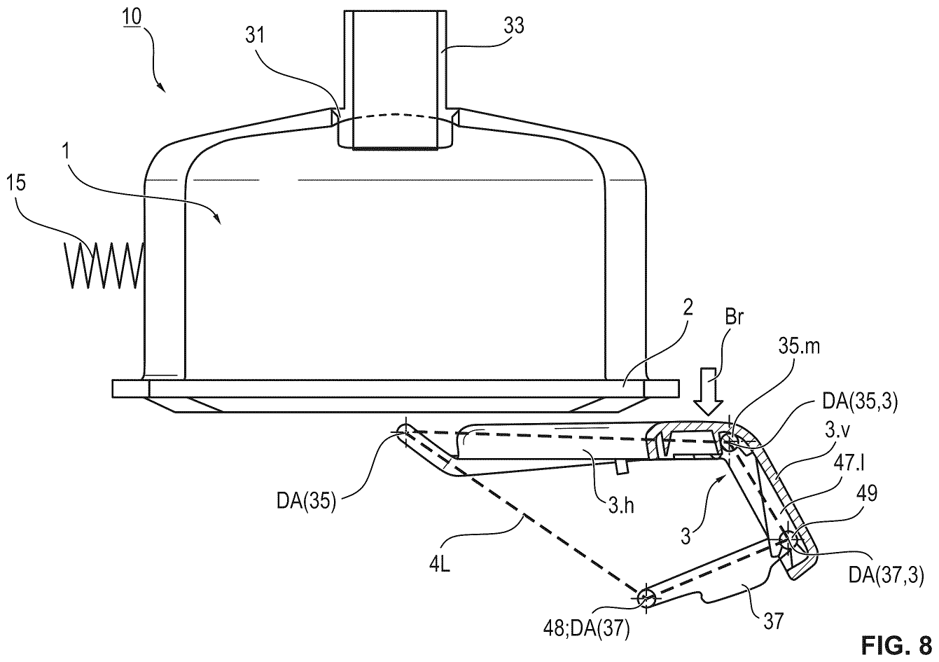

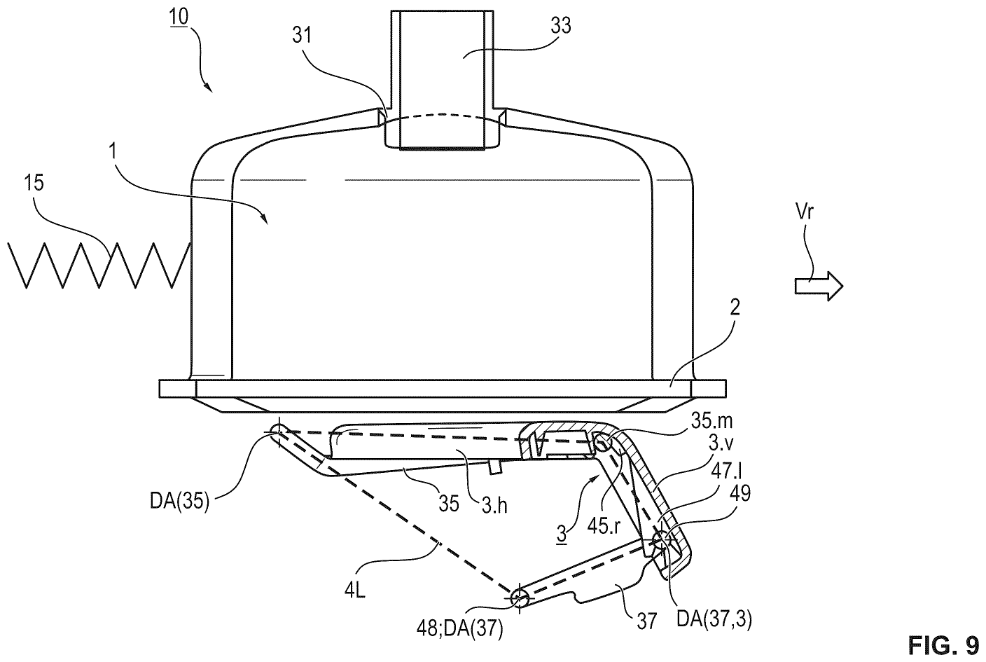

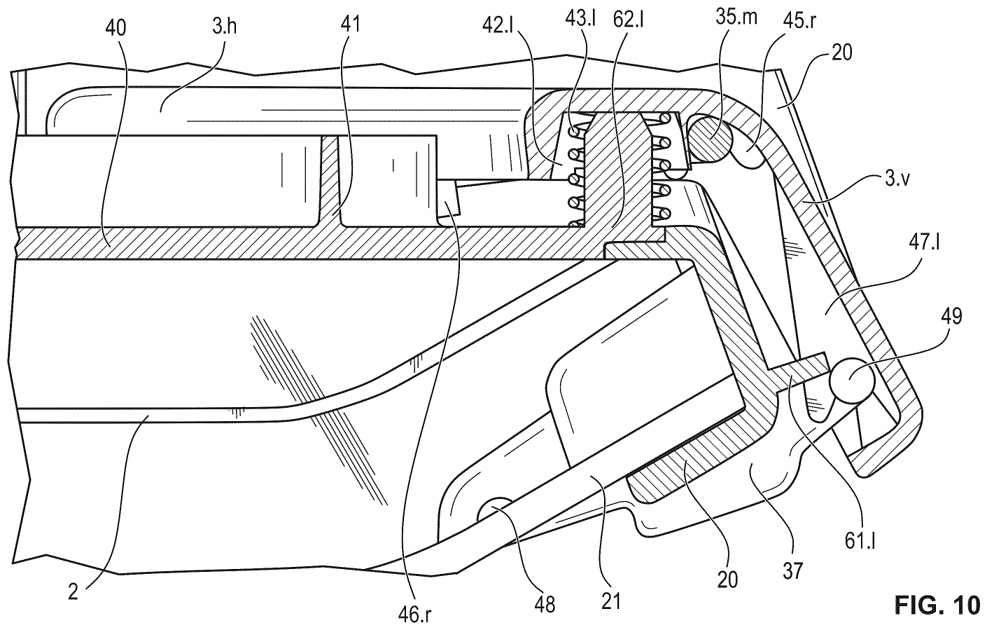

FIG. 4 shows a perspective view of the evaporator unit 10, an actuating unit 3, the locking element 26, and other components, which are described below. FIG. 5 shows the breathing gas humidifier 100, with the evaporator unit 10 and the locking unit 3, 26 omitted. FIG. 6, FIG. 8, and FIG. 9 show the components of FIG. 4 in a sectional view from the side, namely in the sectional plane IV-IV of FIG. 3. FIG. 7 and FIG. 10 show a detail of the representation of FIG. 6 or FIG. 8 and FIG. 9. In FIG. 2 to FIG. 7, the locking unit 3, 26 is in the locking position, in FIG. 8, FIG. 9 and FIG. 10 in the releasing position.

The actuating unit 3 comprises a front rectangular plate 3.v with rounded corners and a rear rectangular plate 3.h with rounded corners. The rear plate 3.h is arranged approximately horizontally when the locking unit 3, 26 is in the locking position, and the front plate 3.v is arranged approximately vertically. The two plates 3.h and 3.v are fixedly connected to each other at a common rounded longitudinal edge, so that an angle of between 100 degrees and 130 degrees occurs between the two plates 3.h and 3.v. The semicircular locking element 26 is fixedly attached to the lower surface of the rear plate 3.h of the actuating unit 3. This lower surface points towards the viewer of FIG. 4. In one embodiment, the two plates 3.h and 3.v and the locking element 26 form a single component, namely the locking unit 3, 26.

A retaining bracket (holding bracket) 35 made of steel or another metal and a retaining plate 37 made of a solid plastic are attached to the housing 20, 21 and in the vicinity of the receptacle for the evaporator unit 10. The retaining bracket 35 acts as the first connecting element. The retaining bracket 35 comprises a U-shaped middle part 35.s, 35.m and two curved connecting rods 35.l, 35.r, which are attached to the free ends of the two legs 35.s of the middle part 35.s, 35.m. The retaining bracket 35 is inherently torsionally rigid. The two connecting rods 35.l and 35.r are rotatably connected to the housing 20, 21. The retaining plate 37 comprises a front connecting pin 49 and a rear connecting pin 48. The retaining bracket 35 is connected to the housing 20, 21 at two connection points, the retaining plate 37 at two further connection points. The retaining bracket 35 is rotatable relative to the housing 20, 21 about an axis of rotation DA(35), the retaining plate 37 about an axis of rotation DA(37). In the embodiment example, the two axes of rotation DA(35) and DA(37) are parallel to each other and horizontal.

A left-hand retaining element 45.l and a right-hand retaining element 45.r are attached to the rear plate 3.h. The two retaining elements (holding elements) 45.l and 45.r are latching connectors that engage around the retaining bracket 35 at two points spaced apart from one another. As a result, the retaining bracket 35 is connected at two connection points to the actuating unit 3 and thus to the locking unit 3, 26. The two retaining elements 45.l and 45.r implement a rear connection, preferably a latching connection, between the retaining bracket 35 and the actuating unit 3. Thanks to the two retaining elements 45.l and 45.r, the retaining bracket 35 is rotatably connected to the actuating unit 3, namely rotatably about an axis of rotation DA(35,3), which is parallel to the axes of rotation DA(35) and DA(37) and extends through the middle part 35.m.

A left-hand retaining element 47.l and a right-hand retaining element 47.r are attached to the front plate 3.v. The two retaining elements 47.l and 47.r are latching connectors that engage around two laterally projecting pins 49.l, 49.r of the retaining plate 37 at two points spaced apart from each other. Thanks to the two retaining elements 47.l and 47.r, the retaining plate 37 is connected to the actuating unit 3 at two further connection points. The two retaining elements 47.l and 47.r implement a front connection, preferably a latching connection, between the retaining plate 37 and the actuating unit 3. Thanks to this front connection, the retaining plate 37 can be rotated relative to the actuating unit 3 about an axis of rotation DA(37,3). In the embodiment example, the four axes of rotation DA(35), DA(37), DA(35,3) and DA(37,3) are parallel to each other. A respective distance occurs between every two parallel axes of rotation.

As already mentioned, the locking unit 3, 26 can be moved from a locking position, which is shown in FIG. 2 to FIG. 6, to a releasing position, which is shown in FIG. 8 and FIG. 9. A left spring 43.l and a right spring 43.r strive to move the locking unit 3, 26 into the locking position and to hold it in the locking position. FIG. 5 schematically shows the two springs 43.l and 43.r and also the guide pins for the two springs 43.l and 43.r. FIG. 4 shows a left circular receptacle 42.l and a right circular receptacle 42.r for the two springs 43.l and 43.r. The locking unit 3, 26 can be moved against the force of the springs 43.l and 43.r from the locking position to the releasing position.

In one implementation, a further left-hand retaining element 46.l and a further right-hand retaining element 46.r are attached to the rear plate 3.h. When the locking unit 3, 26 is in the locking position, there is a gap between the two retaining elements 46.l and 46.r on the one hand and the retaining bracket 35 on the other hand. When the locking unit 3, 26 is in the releasing position, the two retaining elements 46.l and 46.r engage around the retaining bracket 35. The two retaining elements 46.l and 46.r largely prevent the locking unit 3, 26 in the releasing position from moving relative to the receptacle 20, 21, 25 in a direction parallel to the axis of rotation DA(35).

The front latching connectors 47.l, 47.r [axis of rotation DA(37,3)] just described and the rear latching connectors 45.l, 45.r [axis of rotation DA(35,3)] just described as well as the two axes of rotation DA(37) and DA(35) together implement an oblique-angled four-bar linkage 4L. The four-bar linkage 4L guides the locking unit 3, 26 during movement from one position to the other along a curved trajectory that extends in a vertical plane. FIG. 6, FIG. 8 and FIG. 9 show an example of the four-bar linkage 4L.

To move the locking unit 3, 26 from the locking position to the releasing position, a user presses from above on the rear plate 3.h of the actuating unit 3. An arrow indicates a possible actuating direction Br, see FIG. 8. In order to press the actuating unit 3 downwards against the force of the springs 43.l and 43.r and thereby to move the locking unit 3, 26 into the releasing position, it is sufficient for a user to press on the rear plate 3.h at one point, ideally at any point, from above. This desired effect is achieved on the one hand by the four-bar linkage 4L, which provides positive guidance (forced guidance) of the locking unit 3, 26 while moving from one position to the other. On the other hand, the effect is achieved because the retaining bracket 35 is inherently torsionally rigid. Pressure from above on the retaining bracket 35 causes the entire retaining bracket 35 to be rotated about the axis of rotation DA(35) and does not allow the retaining bracket 35 itself to be twisted. As a rule, it is not necessary to press on the rear plate 3.h at two different points at the same time.

A comparison of FIG. 6 with FIG. 8 and FIG. 9 shows the respective orientation of the locking unit 3, 26 in the locking position (FIG. 6) and in the releasing position (FIG. 8, FIG. 9). In the locking position, the rear plate 3.h rises (is angled) slightly as seen in the viewing direction of FIG. 2 and FIG. 3, i.e. towards the housing, such that the locking element 26 rests against the base plate 2. One effect: If a user mistakenly places an evaporator unit 10 on top of the actuating unit 3 without placing it against the locking element 26, the evaporator unit 10 slides off the rear plate 3.h of the actuating unit 3. A user usually notices this. In the releasing position, the rear plate 3.h is arranged approximately horizontally. This makes it easier to pull or push the evaporator unit 10 out of the breathing gas humidifier 100 over the rear plate 3.h and insert the cleaned or a new evaporator unit 10. The inclined or horizontal orientation of the rear plate 3.h is implemented by the configuration of the oblique-angled four-bar linkage 4L.

In one embodiment, an ejector 15 is supported on the front part 20 and/or on the stop unit 25 and strives to move the evaporator unit 10 away from the receptacle 20, 21, 25. The evaporator unit 10 is inserted into the receptacle 20, 21, 25 against the force of the ejector 15. If the locking unit 3, 26 is in the releasing position, the ejector 15 pushes the evaporator unit 10 away from the receptacle 20, 21, 25 in a discharge direction Vr, which is illustrated by a comparison of FIG. 8 with FIG. 9. Preferably, the ejector 15 is configured as a purely mechanical component, for example as a mechanical or pneumatic spring unit.

In particular, this configuration has the following two advantages:
The evaporator unit 10 is only correctly inserted into the receptacle 20, 21, 25 when the locking unit 3, 26 is in the locking position. Only in this case does the locking unit 3, 26 in the locking position prevent the ejector 15 from moving the evaporator unit 10 away from the receptacle 20, 21, 25. If the evaporator unit 10 is not correctly inserted into the receptacle 20, 21, 25, the ejector 15 pushes the evaporator unit 10 away from the receptacle 20, 21, 25, which is clearly visible to a user.
When a user has moved the locking unit 3, 26 into the releasing position and holds it in the releasing position, the ejector 15 causes the evaporator unit 10 to be moved away from the receptacle 20, 21, 25. It is not necessary for the user himself/herself to move the evaporator unit 10 out of the receptacle 20, 21, 25. This makes it possible in many cases to remove the evaporator unit 10 from the receptacle 20, 21, 25 by operating it with one hand, even with a glove.

Even if the locking unit 3, 26 is in the releasing position, the actuating unit 3 remains connected to the retaining bracket 35 and the retaining plate 37. The two springs 43.l and 43.r strive to move the locking unit 3, 26 into the locking position and hold the locking unit 3, 26 in this position in every position of the actuating unit 3. In order to remove an evaporator unit 10, the locking unit 3, 26 must therefore be moved against the force of the springs 43.l and 43.r into the releasing position and be held in this position. This reduces the risk of the evaporator unit 10 unintentionally sliding out of the receptacle.

On the one hand, the four-bar linkage 4L reduces the risk of the locking unit 3, 26 tilting while moving from one position to the other. On the other hand, the four-bar linkage 4L ensures that a left gap and a right gap remain between the moving locking unit 3, 26 and the housing 20, 21, so that the locking unit 3, 26 does not scratch the housing 20, 21. In addition, a gap remains between the locking unit 3, 26 and the evaporator unit 10.

As already mentioned, the two springs 43.l and 43.r strive to move the locking unit 3, 26 into the locking position and to hold it in this position. In the detailed view of FIG. 4, FIG. 7 and FIG. 10, two rear stop elements 60.l and 60.r can be seen at the free end of the rear plate 3.h and two front stop elements 61.l and 61.r can be seen at the front part 20. The rear stop elements 60.l and 60.r touch the stop unit 25 from below when the locking unit 3, 26 is in the locking position. The front plate 3.v touches the front stop elements 61.l and 61.r from below when the locking unit 3, 26 is in the locking position. The stop elements 60.l, 60.r and 61.l, 61.r prevent the springs 43.l and 43.r from pushing the locking unit 3, 26 further upwards.

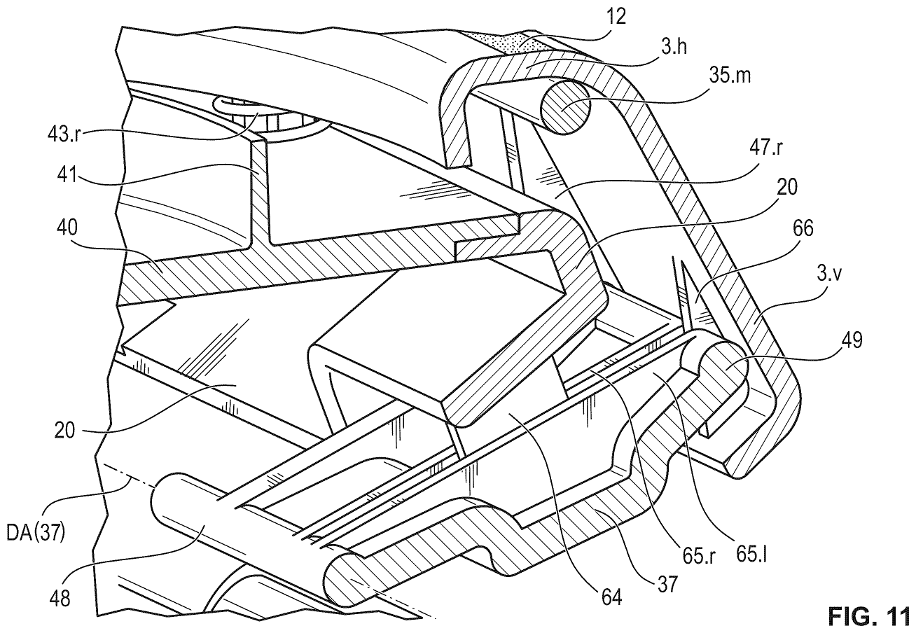

FIG. 11 illustrates how the locking unit 3, 26 is guided while moving from one position to the other. A left guide rail 65.l and a right guide rail 65.r are attached to the retaining plate 37, forming a slot between them. A rectangular projection 64 is attached to the front part 20. This projection 64 engages in the slot between the guide rails 67.l and 67.r. A triangular projection 66 is attached to the front plate 3.v. The projection 66 also engages in the slot between the guide rails 67.l and 67.r. Thanks to this configuration, the locking unit 3, 26 is held with even greater security in a vertical trajectory and guided along this trajectory.

While specific embodiments of the invention have been shown and described in detail to illustrate the application of the principles of the invention, it will be understood that the invention may be embodied otherwise without departing from such principles.

### LIST OF REFERENCE SYMBOLS

| | |
|---|---|
| 1 | Evaporation chamber of the evaporator unit 10, receives water, comprises |
| | the connection 31 for the discharge fluid conveying unit 33 and the |
| | connection 32 for the feed fluid guide unit 30, connected to the base plate |
| | 2 |
| 2 | Circular base plate, connected to the evaporation chamber 1, is in thermal |
| | contact with the heating plate 40 |
| 3 | Actuating unit, comprising the front plate 3.v and the rear plate 3.h, |
| | belongs to the locking unit, which can be moved back and forth between |
| | a locking and a releasing position |
| 3.h | Approximately horizontal rectangular rear plate of the actuating unit 3, |
| | fixedly connected to the front plate 3.v, carries the locking element 26 |
| 3.v | Approximately vertical rectangular front plate of the actuating unit 3, |
| | fixedly connected to the rear plate 3.h |
| 4 | Ventilator, connected to the patient-side fluid coupling unit 7 by means |
| | of the inspiratory fluid guide unit 5, comprises the fluid conveying unit 8, |
| | the housing 22 and the feed connection 19 |
| 5 | Inspiratory fluid guide unit, connects the ventilator 4 to the patient-side |
| | coupling unit 7 |
| 6 | Control unit of the ventilator 4 |
| 7 | Breathing mask on the patient's face P, belongs to the patient-side |
| | coupling unit, connected to the Y-piece 13 |
| 8 | Fluid conveying unit of the ventilator 4 |
| 10 | Evaporator unit, comprising the evaporation chamber 1 and the bottom |
| | plate 2, is locked by the stop unit 25 and the locking element 26, acts as |
| | the container with the liquid to be evaporated and the evaporation |
| | chamber 1. |
| 11 | Expiratory fluid guide unit, leads from the Y-piece 13 into the |
| | environment |
| 12 | Indicator light on the rear panel 3.h, indicates whether an evaporator unit |
| | 10 is correctly inserted in the receptacle 20, 21, 25 or not |
| 13 | Y-piece, connected to the fluid guide units 5, 11 and to the breathing |
| | mask 7 |
| 14 | Valve in the expiratory fluid guide unit 11 |
| 15 | Ejector in the form of a mechanical or pneumatic spring unit or an |
| | electric actuator, rests on the receptacle 20, 21, 25, strives to move the |
| | evaporator unit 10 away from the receptacle 20, 21, 25 |
| 19 | Feed connection of the ventilator 4 |
| 20 | Front part of the receptacle for the evaporator unit 10, connected to the |
| | stop element 25 |
| 21 | Main part of the receptacle for the evaporator unit 10 |
| 22 | Housing of the ventilator 4, fixedly connected to the housing 20, 21 of |
| | the breathing gas humidifier 100 |
| 25 | Semi-circular stop unit for the base plate 2, fixedly connected to the front |
| | part 21, surrounds the base plate 2 |
| 26 | Semicircular locking element for the base plate 2, fixedly connected to |
| | the rear plate 3.h of the actuating unit 3, is part of the locking unit 3, 26, |
| | which can be moved back and forth between a locking and a releasing |
| | position |
| 30 | Feed fluid guide unit |
| 31 | Connection for the discharge fluid guide unit 33 |
| 32 | Connection for the fluid guide unit 30 |
| 33 | Discharge fluid guide unit |
| 34 | Feed opening in the evaporation chamber 1 for feeding in water |
| 35 | Torsionally rigid retaining bracket, rotatable about the axis of rotation |
| | DA(35), comprises the center part 35.s, 35.m and the two connecting rods |
| | 35.1, 35.r, acts as the first connecting element |
| 35.m | Rod of the U-shaped middle part of the retaining bracket 35 |
| 35.1 | Left connecting rod of the retaining bracket 35, attached to a leg 35.s, |
| | rotatable relative to the housing about the axis of rotation DA(35) |
| 35.r | Right connecting rod of the retaining bracket 35, attached to a leg 35.s, |
| | rotatable relative to the housing about the axis of rotation DA(35) |
| 35.s | Leg of the U-shaped middle part of the retaining bracket 35 |
| 37 | Retaining plate, rotatable about the axis of rotation DA(37), acts as a |
| | second connecting element |
| 40 | Retaining plate, comprises a heating plate which is in thermal contact |
| | with the base plate 2 |
| 41 | Retaining ring on the retaining plate 40 |
| 42.1, 42.r | Left or right circular receptacle for spring 43.1, 43.r |
| 43.1, 43.r | Left or right spring, which holds the locking unit 3, 26 in the locking |
| | position |
| 45.1, 45.r | Left or right retaining element on the rear plate 3.h of the actuating unit |
| | 3, engages around the retaining bracket 35, together implementing a |
| | rotatable bearing with the axis of rotation DA (35, 3) and a rear latching |
| | connection |
| 46.1, 46.r | Left or right retaining element on the rear plate 3.h of the actuating unit |
| | 3, engages around the retaining bracket 35, together implementing a |
| | lateral boundary |
| 47.1, 47.r | Left or right retaining element on the front plate 3.v of the actuating unit |
| | 3, engages around the connecting pin 49 on the retaining plate 37, |
| | together implementing a rotatable bearing with the axis of rotation DA |
| | (37, 3) and a front latching connection |
| 48 | Continuous rear connecting pin of the retaining plate 37, rotatable |
| | relative to the housing about the axis of rotation DA(37) |
| 49 | Continuous front connecting pin of the retaining plate 37, is embraced by |
| | the two retaining elements 47.1 and 47.r |
| 60.1, 60.r | Left or right rear stop element at the free end of the rear plate 3.h, touches |
| | the stop unit 25 from below when the actuating unit 3 is in the locking |
| | position |
| 61.1, 61.r | Left or right front stop element at the free end of the front plate 3.h, |
| | touches the front part 20 from below when the locking unit 3, 26 is in the |
| | locking position |
| 62.1, 62.r | Left or right holding pin, holds the spring 43.1 or 43.r |
| 64 | Projection on the front part 20, engages in the slot between the guide rails |
| | 67.1 and 67.r |
| 65.1, 65.r | Left or right guide rail on the retaining plate 37, form a slot between |
| | them, guide the projection 64 |
| 66 | Projection on the front plate 3.v, engages in the slot between the guide |
| | rails 67.1 and 67.r |
| 67 | Guide rail on the retaining plate 37, guides the projection 64 |
| 100 | Breathing gas humidifier, comprising the front part 20, the main part 21, |
| | the stop unit 25, an evaporator unit 10 inserted into the receptacle 20, 21, |
| | 25, the feed fluid guide unit 30 and the discharge fluid guide unit 33 |
| DA(35) | Axis of rotation about which the retaining bracket 35 can be rotated |
| | relative to the housing 20, 21, 25 |
| DA(35, 3) | Axis of rotation about which the actuating unit 3 can be rotated relative to |
| | the retaining bracket 35 |
| DA(37) | Axis of rotation about which the retaining plate 37 can be rotated relative |
| | to the housing 20, 21, 25 |
| DA(37, 3) | Axis of rotation about which the retaining plate 37 can be rotated relative |
| | to the actuating unit 3 |
| Gg | Gas mixture emitted by the ventilator 4, comprising oxygen and |
| | optionally an anesthetic agent, is humidified by the breathing gas |
| | humidifier 100 |
| Gg_{H2O} | Gas mixture Gg, enriched with water vapor, is fed to the patient-side |
| | coupling unit 7 |
| 4L | Quadrilateral of the four-bar linkage |
| P | Patient, connected to the patient-side coupling unit 7 |
| Vr | Discharge direction in which the ejector 15 pushes an evaporator unit 10 |
| | out of the receptacle 20, 21, 25 |

What is claimed is:

1. A breathing gas humidifier for increasing a content of an evaporated liquid in a breathable gas mixture, the breathing gas humidifier comprising:
a container, which is configured to hold a liquid to be evaporated;
a housing with a receptacle;
a heating element;
a locking unit, which is configured to move back and forth relative to the receptacle between a locking position and a releasing position; and
a connection unit,
wherein the breathing gas humidifier is configured such that the container is insertable into the receptacle and is removable from the receptacle,
wherein, with the container correctly inserted into the receptacle, the container is in thermal contact with the heating element,
wherein the heating element is configured to heat the liquid in the inserted container,
wherein the locking unit, in the locking position, holds the container in the receptacle such that movement of the container away from the receptacle is prevented,
wherein the locking unit, in the releasing position, allows removal of the container from the receptacle, and
wherein the connecting unit mechanically connects the locking unit to the housing such that the locking unit is guided along a locking unit trajectory during a movement of the locking unit between the locking position and the releasing position.

2. A breathing gas humidifier according to claim 1,
wherein the connecting unit comprises a first connecting element and a second connecting element,
wherein the first connecting element and the second connecting element are spatially separated from each other,
wherein the first connecting element is pivotably connected to the housing at two spaced-apart connection points and is pivotably connected to the locking unit at two further spaced-apart connection points, and
wherein the second connecting element is pivotably connected to the housing at at least one connection point and is pivotably connected to the locking unit at at least one further connection point.

3. A breathing gas humidifier according to claim 2,
wherein the first connecting element is rotatably connected to the housing at the two spaced-apart connection points such that the first connecting element is rotatably connected to the housing about a first connecting element axis of rotation, and the first connecting element is rotatably connected to the locking unit at the two further spaced-apart connection points such that the first connecting element is rotatably connected to the locking unit about a second connecting element axis of rotation,
wherein the second connecting element is rotatably connected to the housing at the at least one connection point and at another connection point such that the second connecting element is rotatably connected to the housing about a third connecting element axis of rotation, and the second connecting element is rotatably connected to the locking unit about a fourth connecting element axis of rotation,
wherein the four connecting element axes of rotation are pairwise parallel to each other and at a distance from each other, and
wherein the two connecting elements form a four-bar linkage connection of the locking unit to the housing.

4. A breathing gas humidifier according to claim 2,
wherein the first connecting element is torsionally rigid and comprises a U-shaped bracket with two legs and a connecting rod on each one of the two legs, and
wherein the two connecting rods are rotatably connected to the housing.

5. A breathing gas humidifier according to claim 2,
wherein the second connecting element comprises a connecting element side guide element on a connecting element side and the housing comprises a housing side guide element on a housing side, and
wherein the connecting element side guide element corresponds with the housing side guide element such that the connecting element side guide element engages in the housing side guide element and the second connecting element is guided along a connecting element trajectory during the movement of the locking unit between the locking position and the releasing position.

6. A breathing gas humidifier according to claim 2,
wherein the second connecting element comprises a connecting element side guide element on a connecting element side and the locking unit comprises a locking unit side guide element on a locking unit side, and
wherein the connecting element side guide element corresponds to the locking side guide element such that the second connecting element is guided along a connecting element trajectory during the movement of the locking unit between the locking position and the releasing position.

7. A breathing gas humidifier according to claim 2, wherein the first connecting element and the second connecting element are respectively rotatably connected to the locking unit at two spatially spaced-apart connection points.

8. A breathing gas humidifier according to claim 1, wherein the locking unit comprises a plate and the connecting unit connects the locking unit to the housing such that, with the locking unit in the locking position, the plate is in a raised position relative to the receptacle.

9. A breathing gas humidifier according to claim 1, wherein the locking unit comprises a plate and the connecting unit connects the locking unit to the housing such that, with the locking unit in the releasing position, the plate is arranged horizontally and/or the locking unit is located vertically below the receptacle and/or the locking unit is located at an angle and at least partially below the receptacle.

10. A breathing gas humidifier according to claim 1, further comprising an ejector, which ejector is supported on the receptacle and strives to move the container, positioned in the receptacle, away from the receptacle, wherein the locking unit in the releasing position is located completely vertically or obliquely below the container positioned in the receptacle.

11. A breathing gas humidifier according to claim 1, wherein the locking unit in the locking position and the receptacle are configured to together surround the container.

12. A breathing gas humidifier according to claim 1, wherein the locking unit trajectory extends in a plane.

13. A breathing gas humidifier according to claim 4, wherein, with the locking unit in the locking position, the inserted container is located between the receptacle and the U-shaped bracket.

14. A ventilation arrangement for ventilation of a patient, the ventilation arrangement comprising:
a medical device configured to expel a breathable gas mixture;
a fluid guide unit;
a patient-side coupling unit, which is configured to be arranged in and/or on the body of the patient; and
a breathing gas humidifier, the breathing gas humidifier is for increasing a content of an evaporated liquid in a breathable gas mixture, the breathing gas humidifier comprising:
a container, which is configured to hold a liquid to be evaporated;
a housing with a receptacle;
a heating element;
a locking unit, which is configured to move back and forth relative to the receptacle between a locking position and a releasing position; and
a connection unit,
wherein the breathing gas humidifier is configured such that the container is insertable into the receptacle and is removable from the receptacle,
wherein, with the container correctly inserted into the receptacle, the container is in thermal contact with the heating element,
wherein the heating element is configured to heat the liquid in the inserted container,
wherein the locking unit, in the locking position, holds the container in the receptacle such that movement of the container away from the receptacle is prevented,
wherein the locking unit, in the releasing position, allows removal of the container from the receptacle,
wherein the connecting unit mechanically connects the locking unit to the housing such that the locking unit is guided along a locking unit trajectory during a movement of the locking unit between the locking position and the releasing position,
wherein the ventilation arrangement is configured to guide the expelled gas mixture through the fluid guide unit to the patient-side coupling unit, and
wherein the breathing gas humidifier is configured to increase an evaporated liquid content, in the expelled gas mixture, before the gas mixture reaches the patient-side coupling unit.

15. A ventilation arrangement according to claim 14,
wherein the connecting unit comprises a first connecting element and a second connecting element that are spatially separated from each other,
wherein the first connecting element is rotatably connected to the housing at two spaced-apart connection points such that the first connecting element is pivotably connected to the housing about a first connecting element axis, and the first connecting element is pivotably connected to the locking unit at the two further spaced-apart connection points such that the first connecting element is rotatably connected to the locking unit about a second connecting element axis of rotation, and
wherein the second connecting element is pivotably connected to the housing at at least one connection point and at another connection point such that the second connecting element is pivotably connected to the housing about a third connecting element axis, and the second connecting element is pivotably connected to the locking unit about a fourth connecting element axis of rotation,
wherein the four connecting element axes are pairwise parallel to each other and at a distance from each other, and
wherein the two connecting elements form a four-bar linkage connection of the locking unit to the housing.

16. A ventilation arrangement according to claim 15,
wherein the first connecting element is torsionally rigid and comprises a U-shaped bracket with two legs and a connecting rod on each one of the two legs, and
wherein the two connecting rods are rotatably connected to the housing.

17. A ventilation arrangement according to claim 15,
wherein the second connecting element comprises a connecting element side guide element on a connecting element side and the housing comprises a housing side guide element on a housing side,
wherein the connecting element side guide element corresponds with the housing side guide element such that the connecting element side guide element engages in the housing side guide element and the second connecting element is guided along a connecting element trajectory with the movement of the locking unit between the locking position and the releasing position,
wherein the locking unit comprises a locking unit side guide element on a locking unit side, and
wherein the connecting element side guide element corresponds to the locking side guide element such that the second connecting element is guided along the connecting element trajectory with the movement of the locking unit between the locking position and the releasing position.

18. A ventilation arrangement according to claim 14, wherein the locking unit comprises a plate and the connecting unit connects the locking unit to the housing such that, with the locking unit in the locking position, the plate is in a raised position relative to the receptacle and such that with the locking unit in the releasing position, the plate is arranged horizontally and/or the locking unit is located vertically below the receptacle and/or the locking unit is located at an angle and at least partially below the receptacle.

19. A ventilation arrangement according to claim 14, wherein the humidifier further comprises an ejector, which ejector is supported on the receptacle and strives to move the container, positioned in the receptacle, away from the receptacle, wherein the locking unit in the releasing position is located completely vertically or obliquely below the container positioned in the receptacle.

20. A ventilation arrangement according to claim 14, wherein the locking unit trajectory extends in a plane.
